(19) United States
(12) Patent Application Publication
(10) Pub. No.: US 2011/0166332 A1
(43) Pub. Date: Jul. 7, 2011

(54) ENHANCED ANTIBODY AGGREGATE REMOVAL WITH CAPTO ADHERE IN THE PRESENCE OF PROTEIN-EXCLUDED ZWITTERIONS

(75) Inventor: Peter S. Gagnon, San Clemente, CA (US)

(73) Assignee: GE HEALTHCARE BIO-SCIENCES AB, UPPSALA (SE)

(21) Appl. No.: 13/063,221

(22) PCT Filed: Aug. 28, 2009
(86) PCT No.: PCT/SE2009/050968

§ 371 Date: Mar. 10, 2011

Related U.S. Application Data
(60) Provisional application No. 61/191,790, filed on Sep. 12, 2008.

(51) Int. Cl. C07K 1/18

(52) U.S. Cl. 530/416

(57) ABSTRACT

The invention relates to a method for separating at least one non-aggregated protein from a liquid protein preparation by contacting said preparation with a multimodal anion exchanger in the presence of protein-excluded zwitterions at a concentration of 0.25 to 2.5 M.

### CROSS-REFERENCE TO RELATED APPLICATIONS

This application is a filing under 35 U.S.C. §371 and claims priority to international patent application number PCT/SE2009/050968 filed Aug. 28, 2009, published on Mar. 18, 2010 as WO 2010/030222, which claims priority to U.S. provisional patent application No. 61/191,790 filed on Sep. 12, 2008.

### FIELD OF THE INVENTION

This invention relates to methods for enhancing separation of proteins such as IgG monoclonal antibodies by chromatography on multimodal anion exchangers in the presence of zwitterions that are excluded from protein surfaces. In certain embodiments, the invention may permit more effective separation of non-aggregated protein from aggregated protein.

### BACKGROUND OF THE INVENTION

Multimodal anion exchangers are chromatography media that in addition to a positively charged group (amine, quaternary ammonium group etc) comprise a group that interacts with the target molecule via another mechanism than charge-charge interaction. This may be hydrophobic interactions, van der Waals interactions, dipole interactions, cation-pi interactions, hydrogen bonding etc. A useful class of multimodal anion exchangers comprise a positively charged group and in addition an aromatic ring structure. CAPTO™ adhere (trademark owned by GE Healthcare Bio-Sciences AB) belongs to this class and is a multimodal chromatography medium that principally employs a combination of hydrophobic interactions and anion exchange, mediated through a ligand that has a phenyl group and a quaternary amine, as well as other features. Antibody aggregate removal is an important application of CAPTO™ adhere. Commercial literature indicates that its aggregate removal ability is modest however, permitting, for example, reduction in aggregate content from starting levels of a few percent down to final levels ranging from 0.8% to 0.1% [GE Healthcare, Data File 28-9078-88-AA, 2007].

Generic conditions for use of multimodal anion exchangers such as CAPTO™ adhere do not exist. Specific conditions must be developed for each protein. The recommended method development procedure consists of evaluating various combinations of pH and sodium chloride concentration in the hope of identifying conditions that preferentially favor retention of aggregates. The recommended application format is to apply a liquid protein preparation to the column under conditions that allow the non-aggregated protein to pass through the column, while aggregated protein is retained and thereby removed.

A number of small zwitterionic molecules exist which are preferentially excluded from protein surfaces. Glycine is one example of this class of molecules. They have a wide variety of effects on proteins and chromatographic separations. In their zwitterionic forms they have no significant conductivity but exhibit high molar dielectric increments and positive surface tension increments. This combination of characteristics endows them with seemingly self-conflicting abilities that make their effects impossible to predict. They are known to increase protein solubility but may also mediate protein retention on hydrophobic interaction chromatography media. In addition, high dielectric constants weaken ion exchange interactions even though the zwitterionic molecules themselves provide virtually no direct charge competition. Some synthetic zwitterionic compounds, such as MES, Hepes, and Bicine, are employed commonly as buffering agents. The usual operating concentration for buffering applications ranges from about 10 to 50 mM.

### SUMMARY OF THE INVENTION

The present invention relates to a method of separating at least one intact non-aggregated protein (e.g. an IgG antibody) from a liquid preparation by contacting said preparation with a multimodal anion exchanger such as CAPTO™ adhere in the presence of one or more species of protein-excluded zwitterions at a combined concentration greater than 0.25 M. Applicant surprisingly found that this method substantially enhances the ability of the multimodal anion exchanger to remove protein aggregates from a protein preparation.

Alternatively, said non-aggregated protein is separated from a liquid preparation by contacting said preparation with a multimodal anion exchanger and subsequently contacting said support with a liquid containing protein-excluded zwitterions at a combined concentration greater than 0.25 M.

In some embodiments, practicing the invention may permit removal of aggregates to lower levels than can be achieved in the absence of the invention, such as below 0.8-0.1%.

In some embodiments, practicing the invention may permit effective aggregate removal from a larger amount of protein, measured as mg of protein per mL of the multimodal anion exchanger, than can be achieved in the absence of the invention.

In some embodiments, practicing the invention may permit aggregate removal to a lower level and from a larger amount of protein than can be achieved in the absence of the invention.

In some embodiments, practicing the invention may permit aggregates to be removed effectively from protein preparations that cannot be accommodated in the absence of the invention.

In some embodiments, practicing the invention may permit effective application of a 2-step or multistep purification process comprising affinity chromatography on protein A media followed by chromatography on a multimodal anion exchanger (e.g. CAPTO™ adhere), with proteins (e.g. IgG antibodies) that are not adequately accommodated by a 2-step or multistep purification process with the same chromatography materials in the absence of the invention. The protein A media may comprise modified protein A ligands like in the commercial product MABSELECT SURE™ (trademark owned by GE Healthcare Bio-Sciences AB).

In some embodiments, the protein preparation may be applied to the multimodal anion exchanger (e.g. CAPTO™ adhere) under conditions that permit the binding of non-aggregated protein and aggregated protein, with separation of non-aggregated protein being achieved subsequently by application of an elution gradient. This mode of chromatography is often referred to as bind-elute mode.

In some embodiments, the protein preparation may be applied to the multimodal anion exchanger (e.g. CAPTO™ adhere) under conditions that prevent the binding of non-aggregated protein, while selectively binding or retaining aggregates. This mode of application is often referred to as flow-through mode. Bound aggregates may be removed subsequently from the column by means of a cleaning step.

The invention may be practiced in combination with one or more other separation methods, including but not limited to protein A and other forms of affinity chromatography.

Additional objects and advantages of the invention will be set forth in part in the description which follows, and in part will be obvious from the description, or may be learned by practice of the invention. The objects and advantages of the invention will be realized and attained by means of the elements and combinations specified in the claims.

It is to be understood that both the foregoing general description and the following detailed description are exemplary and explanatory only and are not restrictive of the invention, as claimed.

### BRIEF DESCRIPTION OF THE DRAWINGS

FIG. 1 shows the enhanced antibody aggregate removal under bind-elute conditions with CAPTO™ adhere in the presence of protein-excluded zwitterions. The chromatogram on the left illustrates partial aggregate separation by CAPTO™ adhere. The chromatogram on the right illustrates improved aggregate separation resulting from application of the invention. The solid line represents the UV absorbance trace. The coarse dashed line represents the pH trace. The fine dashed line represents conductivity. Refer to Examples 1 and 2 for procedural details.

### DETAILED DESCRIPTION OF THE INVENTION

### Definitions

Terms are defined so that the invention may be understood more readily. Additional definitions are set forth throughout the detailed description.

“CAPTO™ adhere” refers to a chromatography support employing the ligand N-benzyl-N-methyl ethanolamine immobilized on porous agarose particles.

“Salt” refers to an aqueous-soluble ionic compound formed by the combination of negatively charged anions and positively charged cations. The anion or cation may be of organic or inorganic origin. Examples include but are not limited to sodium chloride.

“Protein-excluded zwitterions” refers to small molecules that bear a balance of positive and negative charges at a given pH such that they do not contribute significantly to the conductivity of the solution in which they are dissolved. Such substances are generally characterized by high molar dielectric increments and positive surface tension increments. The term “protein-excluded” arises from the fact that these molecules are repelled from protein surfaces. Examples of protein-excluded zwitterions may include naturally occurring, chemically modified, or exclusively synthetic compositions, including but not limited to glycine, betaine, taurine, tauro-betaine, morpholinoethanesulfonic acid (MES), hydroxyethylpiperazinesulfonic acid (HEPES), and N,N-Bis(2-hydroxyethyl)glycine (BICINE).

“Buffering compound” refers to a chemical compound employed for the purpose of stabilizing the pH of an aqueous solution within a specified range. Phosphate is one example of a buffering compound. Other common examples include but are not limited to compounds such as acetate, citrate, borate, MES, Tris, and HEPES, among many others.

“Buffer” refers to an aqueous formulation comprising a buffering compound and other components required to establish a specified set of conditions to mediate control of a chromatography method. The term “equilibration buffer” refers to a buffer formulated to create the initial operating conditions. “Wash buffer” refers to a buffer formulated to displace unbound contaminants from a chromatography support. “Elution buffer” refers to a buffer formulated to displace the one or more components from the chromatography support.

Protein refers to any type of protein, glycoprotein, phosphoprotein or protein conjugate. Antibodies constitute a commercially important class of proteins. Other proteins of commercial interest are peptides, insulin, erythropoietin, interferons, enzymes, plasma proteins etc. The molecules of the protein may comprise several subunit chains joined together by e.g. disulfide bonds. In the case of proteins naturally occurring as multimers, each such multimer is here considered as a protein molecule.

Antibody refers to any immunoglobulin molecule, antigen-binding immunoglobulin fragment or immunoglobulin fusion protein, monoclonal or polyclonal, derived from human or other animal cell lines, including natural or genetically modified forms such as humanized, human, chimeric, synthetic, recombinant, hybrid, mutated, grafted, and in vitro generated antibodies. Commonly known natural immunoglobulin antibodies include IgA (dimeric), IgG, IgE, IgG and IgM (pentameric).

“IgG” refers to any immunoglobulin G molecule, monoclonal or polyclonal, derived from human or other mammalian cell lines, including natural or genetically modified forms such as humanized, human, chimeric, synthetic, recombinant, hybrid, mutated, grafted, and in vitro generated antibodies.

“Protein aggregate” refers to an association of at least two protein molecules. The association may be either covalent or non-covalent without respect to the mechanism by which the protein molecules are associated. The association may be direct between the protein molecules or indirect through other molecules that link the protein molecules together. Examples of the latter include but are not limited to disulfide linkages with other proteins, hydrophobic associations with lipids, charge associations with DNA, affinity associations with leached protein A, or mixed mode associations with multiple components.

“Protein preparation” refers to any composition containing a non-aggregated protein (e.g. an IgG antibody). Said preparation may contain protein fragments and/or aggregates. Other proteins and other contaminants, potentially including but not limited to nucleic acids, endotoxins, and virus particles may also be present. Said preparation may be a liquid solution, optionally comprising suspended particles.

As it relates to the invention herein, the term “bind-elute mode” refers to an operational approach to chromatography in which the buffer conditions are established so that the non-aggregated protein, aggregates, and some contaminants bind to the column upon application, with fractionation of non-aggregated protein being achieved subsequently by modification of the buffer conditions. Fractionation is most commonly achieved by applying an elution gradient, in which the concentration of one or more buffer components, or conditions such as pH, are increased or decreased. The increase or decrease may be essentially continuous, as in the case of so-called linear gradients, or incremental, as in the case of so-called step gradients.

As it relates to the invention herein, the term “flow-through mode” refers to an operational approach to chromatography in which the buffer conditions are established so that the intact non-aggregated protein flows through the column upon application while contaminants are selectively bound or retained, thus achieving their removal.

“Preparative applications” refers to situations in which the invention is practiced for the purpose of separating non-aggregated protein for research, diagnostic, or therapeutic applications. Such applications may be practiced at any scale, ranging from milligrams to kilograms of protein per batch.

The term “multimodal ion exchanger” as used in this application is not intended to encompass the area of “affinity media”. With affinity media is meant a matrix having a ligand that interacts through biospecific interactions with a target species.

### Multimodal Anion Exchanger

In one embodiment, a multimodal anion exchanger is used. Multimodal anion exchangers are chromatography media that in addition to a positively charged group (amine, quaternary ammonium group etc) comprise a group that interacts with the target molecule via another mechanism than charge-charge interaction. This may be hydrophobic interactions, van der Waals interactions, dipole interactions, cation-pi interactions, hydrogen bonding etc. The multimodal anion exchanger is preferably prepared from a base matrix (porous beads, membrane etc.) to which ligands (also called substituents) have been covalently attached to provide the desired functionality. The base matrix can comprise a polymer material such as a polysaccharide (e.g. agarose or cellulose) or a synthetic polymer (derived e.g. from methacrylate or styrenic monomers).

In one embodiment the multimodal anion exchanger comprises a positively charged group (preferably an amine or a quaternary ammonium ion) and an aromatic ring structure. These functionalities can be accommodated either in separate substituents/ligands as a more or less stochastic mixture of ligands or with both functionalities present in the same substituent/ligand. In the latter case, a preferred ligand structure is described by the following formula,

R₁—R₂—N(R₃)—R₄—R₅

wherein

R₁ is a substituted or non-substituted phenyl group;

R₂ is a hydrocarbon chain comprising 0-4 carbon atoms;

R₃ is a hydrocarbon chain comprising 1-3 carbon atoms;

R₄ is a hydrocarbon chain comprising 1-5 carbon atoms; and

R₅ is OH or H.

This ligand is preferably coupled to the matrix via the nitrogen atom. Particularly preferred ligands are N-benzyl-N-methyl ethanolamine and N,N-dimethyl benzylamine.

CAPTO™ adhere is a commercially available multimodal anion exchanger. According to the manufacturer: “CAPTO™ adhere is based on a rigid agarose matrix that allows high fluid velocities to be used. The highly cross-linked agarose base matrix gives the medium high chemical and physical stability. The CAPTO™ adhere ligand, N-benzyl-N-methylethanolamine, exhibits many functionalities for interaction. The most pronounced are ionic interaction, hydrogen bonding and hydrophobic interaction.”

The invention may be practiced in a packed bed column, a fluidized/expanded bed column containing the multimodal anion exchanger, and/or a batch operation where the multimodal anion exchanger (e.g. CAPTO™ adhere) is mixed with the solution for a certain time.

One embodiment employs a multimodal anion exchanger (e.g. CAPTO™ adhere) packed in a column

One embodiment employs a multimodal anion exchanger, packed in a column of about 5-10 mm internal diameter and a height of about 5-50 mm, for evaluating the effects of various buffer conditions on the binding and elution characteristics of a particular protein preparation. The column may comprise multimodal anion exchanger resin packed in one or more wells of a multiwell filter plate.

One embodiment employs a multimodal anion exchanger, packed in columns of any dimensions required to support preparative applications. Column diameter may range from 1 cm to more than 1 meter, and column height may range from 5 cm to more than 30 cm depending on the requirements of a particular application.

Appropriate column dimensions can be determined by the skilled artisan.

In one embodiment the multimodal anion exchanger is in the form of a membrane, preferably accommodated in a membrane adsorber device.

Protein preparations to which the invention can be applied may include unpurified or partially purified antibodies (e.g. IgG) from natural, synthetic, or recombinant sources. Unpurified antibody preparations may come from various sources including, but not limited to, plasma, serum, ascites fluid, milk, plant extracts, bacterial lysates, yeast lysates, or conditioned cell culture media. Partially purified preparations may come from unpurified preparations that have been processed by at least one chromatography, precipitation, other fractionation step, or any combination of the foregoing. The chromatography step or steps may employ any method, including but not limited to size exclusion, affinity, anion exchange, cation exchange, protein A affinity, hydrophobic interaction, immobilized metal affinity chromatography, or hydroxyapatite chromatography. The precipitation step or steps may include salt or PEG precipitation, or precipitation with organic acids, organic bases, or other agents. Other fractionation steps may include but are not limited to crystallization, liquid:liquid partitioning, or membrane filtration.

In preparation for contacting the protein preparation with the multimodal anion exchanger column, it is usually necessary to equilibrate the chemical environment inside the column. This is accomplished by flowing an equilibration buffer through the column to establish the appropriate pH, conductivity, concentration of salts; and/or the identity and concentration of protein-excluded zwitterions.

The equilibration buffer for applications conducted in bind-elute mode may include any of a wide range of options depending on the binding requirements of a particular protein. The equilibration buffer will normally include a buffering compound to confer adequate pH control. Buffering compounds may include but are not limited to MES, HEPES, BICINE, imidazole, Tris, phosphate, citrate, or acetate, or some mixture of the foregoing or other buffers. The concentration of a buffering compound in an equilibration buffer commonly ranges from 10 to 50 mM. The pH of the equilibration buffer may range from about pH 4.0 to pH 9.5. The equilibration buffer may also contain a salt and/or protein-excluded zwitterion at a specified concentration, such as 0.25-2.5 or 0.75-2.5 M protein-excluded zwitterion.

The protein preparation may also be equilibrated to conditions compatible with the column equilibration buffer in order to facilitate effective binding.

After the column and protein preparation have been prepared, the protein preparation may be contacted with the column. The protein preparation may be applied at a linear flow velocity in the range of, but not limited to, about 50-600 cm/hr. The appropriate flow velocity can be determined by the skilled artisan.

After the sample has been applied, the column is washed to remove unbound contaminants.

The wash buffer may serve the additional purpose of re-equilibrating the column to conditions suitable for practicing the invention. In some embodiments, the wash buffer may contain protein-excluded zwitterions at a concentration of 0.25-2.5 or 0.75-2.5 M.

Following the wash step, non-aggregated protein is selectively eluted from the column. In one embodiment, the concentration of protein-excluded zwitterions is 0.25-2.5 or 0.75-2.5 M at some point during the elution.

In one embodiment, the concentration of protein-excluded zwitterions is increased during elution.

In one embodiment, the concentration of protein-excluded zwitterions is constant during elution.

In one embodiment, the concentration of protein-excluded zwitterions is decreased during elution.

The useful pH range of a particular compound for practicing the invention is determined by its pH titration characteristics. Glycine is in its zwitterionic form at pH values ranging from about pH 4 to about pH 8. Betaine is in its zwitterionic form at pH values ranging from about pH 4 to about pH 10. Taurine is in its zwitterionic form at pH values ranging from about pH 3 to about pH 8. In one embodiment the pH value is chosen so that the compound is in its zwitterionic form.

In one embodiment the protein-excluded zwitterion is selected from the group consisting of glycine, betaine, taurine, tauro-betaine, MES, HEPES and BICINE. In another embodiment, the protein-excluded zwitterion is selected from the group consisting of betaine, taurine, tauro-betaine, MES, HEPES and BICINE. In one embodiment, the protein-excluded zwitterion is glycine.

After elution, the column may optionally be cleaned and re-used, or stored in an appropriate agent for later re-use.

In one embodiment of the flow-through mode, the non-aggregated protein flows through the column and are collected, while aggregated proteins bind to the column. The protein preparation is followed with a wash buffer, usually of the same composition as the equilibration buffer. This displaces remaining non-aggregated protein from the column so that it can be collected. Retained contaminants, including aggregates, may optionally be removed from the column with a cleaning buffer. The column may optionally be re-used, or stored in an appropriate agent for later re-use.

The present invention may be combined with other separation methods to achieve higher levels of purification, if necessary. The invention may be practiced at any point in a sequence of 2 or more purification methods. Examples of methods include, but are not limited to, other methods commonly used for purification of antibodies and other proteins, such as size exclusion chromatography, protein A and other forms of affinity chromatography, anion exchange chromatography, cation exchange chromatography, hydrophobic interaction chromatography, immobilized metal affinity chromatography, hydroxyapatite chromatography, precipitation, crystallization, liquid:liquid partitioning, and various filtration methods. It is within the purview of one of ordinary skill in the art to develop appropriate conditions for the various methods and integrate them with the invention herein to achieve the necessary purification of a particular protein.

All cited references are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, chromatography conditions, and so forth used in the specification and claims are to be understood as being modified in all instances by the term “about.” Accordingly, unless indicated to the contrary, the numerical parameters set forth in the specification and attached claims are approximations that may vary depending upon the desired performance sought to be obtained by the present invention.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with the true scope and spirit of the invention being indicated by the following claims.

It is well known in the art of protein separation that considerable variation in chromatographic behavior is encountered from one protein preparation to another. This includes variation in the composition and proportion of contaminant proteins, intact target protein, target protein fragments, and target protein aggregates that reside within various preparations, as well as variation in the individual retention characteristics of the various sample constituents. This makes it necessary to customize the buffer conditions to apply the invention to its best advantage in each situation. This may involve adjustment of pH, the concentration of salts, the concentration of buffering components, and the identity and concentration of the protein-excluded zwitterion. Appropriate levels for the various parameters and components can be determined systematically by a variety of approaches. The following examples are offered for illustrative purposes only.

### EXAMPLES

### Example 1

Development of an experimental control by initial screening without practicing the invention. A 1 mL Hi-Trap column (7×25 mm) containing CAPTO™ adhere was equilibrated to 20 mM Tris, 20 mM Hepes, 20 mM MES, pH 9.0 at a flow rate of 1 mL/min About 0.27 mL of sample, containing about 5 mg of protein A purified IgG was diluted with equilibration buffer to a final volume of 2 mL, and injected onto the column. The column was washed with equilibration buffer, then with 20 mM Tris, 20 mM Hepes, 20 mM MES, 50 mM sodium chloride, pH 9.0. The column was eluted in a 20 column volume (CV) linear gradient to 20 mM Tris, 20 mM Hepes, 20 mM MES, 50 mM sodium chloride, pH 4.5. The column was cleaned with 6 M guanidine, pH 5.

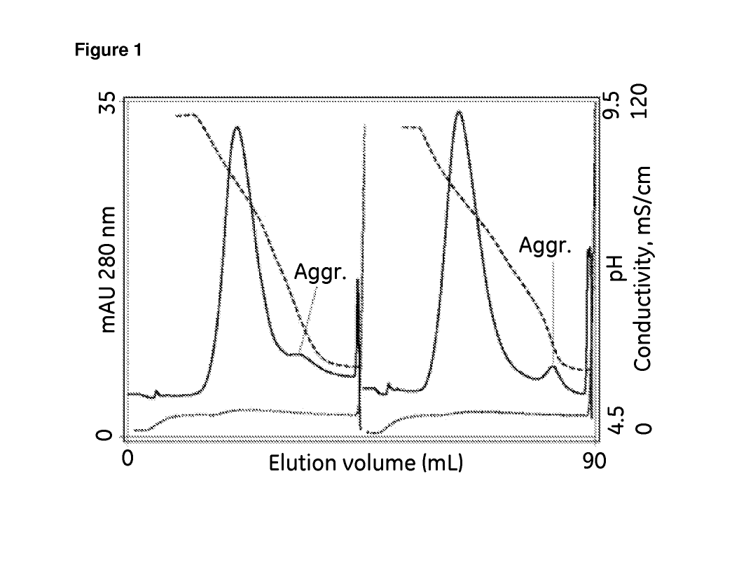

Initial screening by practicing the invention. Another run was conducted under identical conditions except that the elution buffer additionally contained 1 M glycine. The elution profile is compared with the profile from the experimental control in FIG. 1. Comparison shows that the invention enables superior fractionation of a component that elutes later than the main antibody peak. Analytical size exclusion chromatography revealed that the invention reduced aggregate content beneath the level of detection.

It will be understood by the person of ordinary skill in the art how to translate results such as those described above to flow-through conditions, if desired, as well as how to optimize and scale up the method, in either bind-elute or flow-through mode. It will also be understood by such persons that other approaches to method development, such as but not limited to high-throughput robotic systems, can be employed to determine the conditions that most effectively embody the invention for a particular protein.

It will be apparent to the person of ordinary skill that the invention may have a beneficial effect on removal of other contaminants, such as contaminant proteins (e.g. host cell proteins), nucleic acids, endotoxin, virus, and leached protein A.

1. A method for separating at least one non-aggregated protein from a liquid preparation by contacting said preparation with a multimodal anion exchanger in the presence of protein-excluded zwitterions at a concentration of 0.25 to 2.5 M.

2. The method of claim 1, wherein the multimodal anion exchanger comprises ligands defined by the following formula
R₁—R₂—N(R₃)—R₄—R₅
wherein
R₁ is a substituted or non-substituted phenyl group;
R₂ is a hydrocarbon chain comprising 0-4 carbon atoms;
R₃ is a hydrocarbon chain comprising 1-3 carbon atoms;
R₄ is a hydrocarbon chain comprising 1-5 carbon atoms; and
R₅ is OH or H.

3. The method of claim 1, where the ligands of the multimodal anion exchanger comprise N-benzyl-N-methyl ethanolamine or N,N-dimethyl benzylamine.

4. The method of claim 2, wherein the ligands are attached to the matrix via the nitrogen atom.

5. The method of claim 1, wherein the ligand is N-benzyl-N-methyl ethanolamine on a membrane base matrix.

6. The method of claim 1, wherein the ligand is N-benzyl-N-methyl ethanolamine on an agarose base matrix.

7. The method of claim 1, wherein the multimodal anion exchanger is CAPTO™ adhere.

8. The method of claim 1, wherein the protein-excluded zwitterion is glycine.

9. The method of claim 1, wherein the method is part of a multistep purification process comprising affinity chromatography followed by chromatography with a multimodal anion exchanger.

10. The method of claim 8, wherein the affinity chromatography step is carried out using Protein A media.

11. The method of claim 8, wherein affinity chromatography step is carried out using media comprising a modified protein A ligand.

12. The method of claim 9, wherein the affinity chromatography step is carried out using MABSELECT SURE™.

13. The method of claim 1, wherein the protein is an antibody.

14. The method of claim 11, wherein the antibody is an IgG antibody.

15. The method of claim 11, wherein the antibody is a monoclonal antibody.
